(19) United States
(12) Patent Application Publication
(10) Pub. No.: US 2026/0159754 A1
(43) Pub. Date: Jun. 11, 2026

(54) BI2S3/ZNS COMPOSITES COMPRISING S-BI DOUBLE DEFECT AND PREPARATION METHOD THEREOF

(71) Applicant: JIANGSU UNIVERSITY, Jiangsu (CN)

(72) Inventors: Haixia LIU, Jiangsu (CN); Xinlin LIU, Jiangsu (CN); Peng LYU, Jiangsu (CN); Liguang TANG, Jiangsu (CN); Jiaqi WANG, Jiangsu (CN); Yangrui XU, Jiangsu (CN); Guosheng ZHOU, Jiangsu (CN); Yu GU, Jiangsu (CN); Ziyang LU, Jiangsu (CN); Jie JIN, Jiangsu (CN)

(73) Assignee: JIANGSU UNIVERSITY, Jiangsu (CN)

(21) Appl. No.: 18/864,545

(22) PCT Filed: Jul. 19, 2024
(86) PCT No.: PCT/CN2024/106476

§ 371 Date: Nov. 11, 2024

(30) Foreign Application Priority Data
Sep. 22, 2023 (CN) 202311232657.6

(51) Int. Cl. C09K 11/74

(52) U.S. Cl. CPC C09K 11/7414

(57) ABSTRACT

This application discloses Bi₂S₃/ZnS composites comprising S—Bi double defect and its preparation method. The preparation method includes: bismuth nitrate pentahydrate is dissolved in mannitol solution to obtain solution A; zinc chloride is dissolved in water to obtain solution B; solution A is mixed with solution B, L-cysteine is added, and then obtained by hydrothermal reaction. The method described in this application can obtain Bi₂S₃/ZnS composites containing both cationic Bi defects and anionic S defects. The composite material can inhibit the recombination of photogenerated electron-hole pairs while promoting the photogenerated charge transfer rate, and effectively improve the ability of photocatalytic reduction of carbon dioxide.

### CROSS REFERENCE

The present application claims priority to Chinese patent application No. 202311232657.6, filed to the Chinese Patent Office on Sep. 22, 2023, entitled “Bi₂S₃/ZnS composite photocatalysts comprising S—Bi double defect and preparation method thereof”, the entire disclosure of which is incorporated herein by reference.

### TECHNICAL FIELD

The application relates to the field of composite material, and in particular, to Bi₂S₃/ZnS composites comprising S—Bi double defect and preparation method thereof.

### BACKGROUND ART

The massive consumption of fossil fuels has led to a sharp increase in carbon dioxide emissions in the atmosphere, triggering global warming and energy crisis, which has seriously affected the sustainable development of human beings. Solving the energy crisis and global warming is a key issue that cannot be ignored at present. Solar energy is a renewable energy source with sufficient energy reserves. Using solar energy as the driving force, semiconductor as the medium, and carbon dioxide as the raw material, the conversion of this greenhouse gas into a high value-added hydrocarbon fuel can not only solve the problem of global warming at the source, but also provide a new idea for the development of new energy sources. Among semiconductor photocatalysts, metal sulfides such as ZnS and Bi₂S₃ are widely used for photocatalytic reduction of carbon dioxide due to their suitable reduction potential, wide visible light absorption range and fast electron transfer rate. However, the efficiency of photogenerated charge transfer and the recombination of photogenerated electron-hole pairs have always restricted the performance of photocatalysts. It is particularly important to improve the photoelectric properties and enhance the photocatalytic activity of materials by means of defect engineering.

Current studies have shown that defect engineering can effectively regulate the internal electronic and geometric structure of the photocatalyst, thereby improving the catalytic activity of the catalyst. According to the different atomic charges in the compound, the defects can be divided into anionic defects and cationic defects. Among them, anionic defects and cationic defects play different roles in photocatalytic materials. Cationic defects tend to promote the transfer rate of photogenerated charges. Anionic defects are more conducive to inhibiting the recombination of photogenerated electron-hole pairs. The photocatalytic performance of the material can be effectively improved by regulating different defects.

However, there are no reports on the effective combination of cation defects and anion defects to achieve photocatalytic reduction of carbon dioxide to produce carbon monoxide and methane.

### SUMMARY OF THE INVENTION

Based on the above technical problems, this application provides a preparation method of photocatalyst.

The specific scheme of this application is as follows.

This application proposes a preparation method of Bi₂S₃/ZnS composites comprising S-Bi double defect. The preparation method includes: Bismuth nitrate pentahydrate is dissolved in mannitol solution to obtain solution A; Zinc chloride is dissolved in water to obtain solution B; Solution A is mixed with solution B, L-cysteine is added, and then obtained by hydrothermal reaction.

The method described in this application uses L-cysteine as a sulfur source to form an anionic S defect on ZnS, which can effectively promote the separation of photogenerated electron-hole pairs and improve the utilization of photogenerated charges. And mannitol is added as a solvent during the synthesis process to achieve effective regulation of metal cation Bi defects. Cationic Bi defects are formed on Bi₂S₃, which will not affect S defects and increased the transfer rate of photogenerated electrons.

Wherein, in solution A, the molar concentration of bismuth nitrate pentahydrate is 1-5 mmol:30 ml.

Wherein, the concentration of mannitol solution is 0.1-0.5M.

Wherein, in solution B, the molar concentration of zinc chloride is 0.3-6 mmol:20 ml.

Wherein, the molar ratio of bismuth nitrate pentahydrate to zinc chloride is 2:0.3-6, and the molar ratio of bismuth nitrate pentahydrate to L-cysteine is 2:20-30.

Wherein, the molar ratio of bismuth nitrate pentahydrate to zinc chloride is 2:3, and the molar ratio of bismuth nitrate pentahydrate to L-cysteine is 2:25.

Wherein, the hydrothermal reaction temperature is 160-200° C., and the hydrothermal reaction time is 18-26 h.

Wherein, it also includes washing and drying the hydrothermal products, the drying temperature is 50-80° C. and the drying time is 10-20 h.

Wherein, zinc chloride is dissolved in water and ultrasonicized for 10-40 min, and stirred for 10-40 min to obtain solution B.

This application also provides a Bi₂S₃/ZnS composite comprising S—Bi double defect, which is prepared by any of the above methods.

The beneficial effects of this application are as follow.

This application provides a preparation method that can obtain Bi₂S₃/ZnS composites comprising S—Bi double defect. The method can form an anionic Bi defect without affecting the S defect.

The Bi₂S₃/ZnS composites comprising S—Bi double defect are used for photocatalytic reduction of carbon dioxide. The regulation based on the two defects can promote the transfer rate of photogenerated charge and inhibit the recombination of photogenerated electron-hole pairs. At the same time, the existence of multi-site defects effectively increases the specific surface area of the composite material and provides more reaction sites for the photocatalytic reduction of carbon dioxide. In addition, the defects also act as electron enrichment centers to participate in the carbon dioxide reduction reaction and convert it into high value-added hydrocarbons such as carbon monoxide and methane. Compared with the single defect Bi₂S₃/ZnS composites, its photocatalytic performance has been significantly improved, which provides a green, environmentally friendly and efficient treatment method and material basis for effectively solving the problem of carbon dioxide emission pollution.

### BRIEF DESCRIPTION OF THE DRAWINGS

FIG. 1 is the SEM image of different samples, wherein (a) is Bi₂S₃ obtained by Comparative Example 5, (b) is ZnS obtained by Comparative Example 4, and (c) is Bi₂S₃/ZnS composite obtained by Embodiment 1.

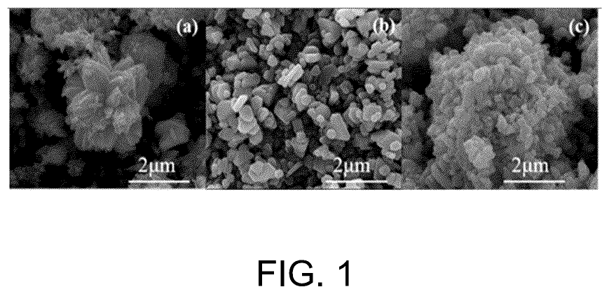

FIG. 2 is the TEM, electron diffraction and HRTEM spectra of Bi₂S₃/ZnS composites obtained by Embodiment 1, wherein (d) is the TEM image, (e) is the electron diffraction image, (f) is the HRTEM image.

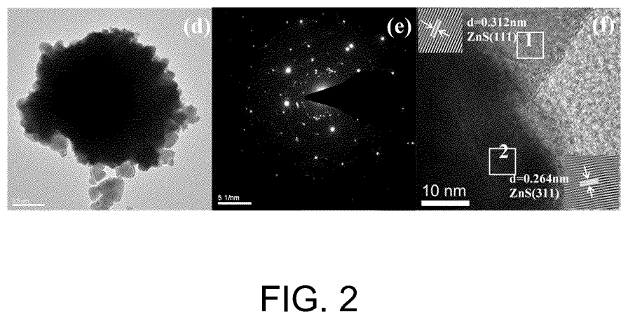

FIG. 3 is the XRD spectra of different samples corresponding to the Bi₂S₃ obtained by Comparative Example 5, the ZnS obtained Comparative Example 4 and the Bi₂S₃/ZnS composite obtained by Embodiment 1.

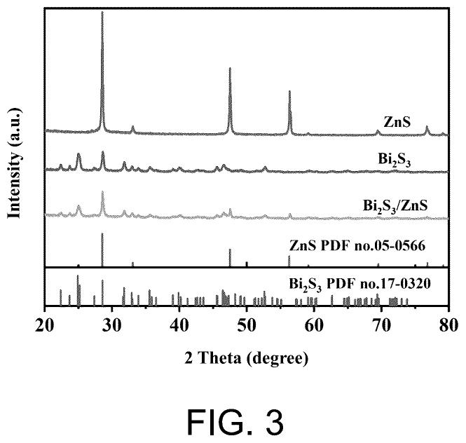

FIG. 4 is the ESR spectra of different samples, wherein (a) is the ESR spectra of BI2S3 under different solvents in Comparative Example 5 and Comparative Example 6 respectively; (b) is the ZnS obtained Comparative Example 4, the D-Bi₂S₃ obtained by Comparative Example 5, and the Bi₂S₃/ZnS composite obtained by Embodiment 1 respectively;

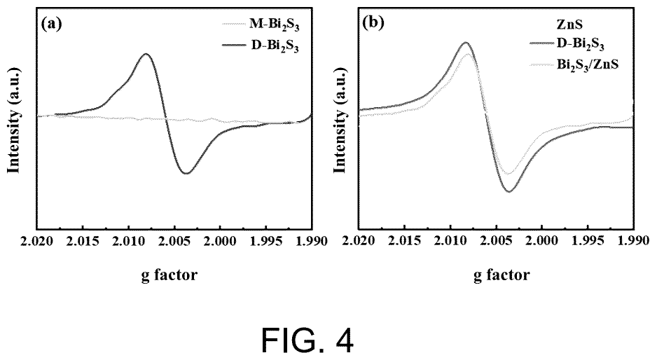

FIG. 5 is the UV-Vis diffuse reflectance spectra of different samples corresponding to the Bi₂S₃ obtained by Comparative Example 5, the ZnS obtained Comparative Example 4 and the Bi₂S₃/ZnS composite obtained by Embodiment 1 respectively.

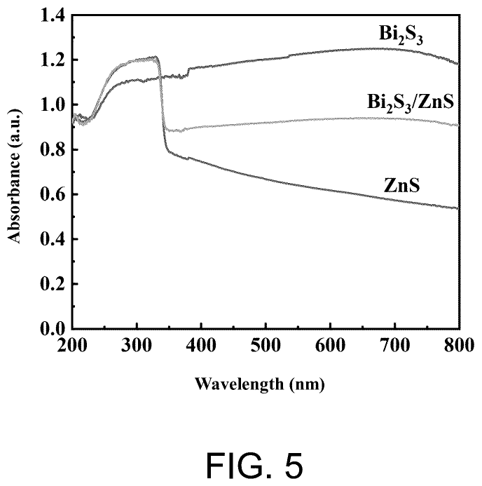

FIG. 6 (a) is the cycle characteristics of Bi₂S₃/ZnS composite for CO₂ reduction obtained in Embodiment 1, and (b) is the XRD diagram before and after the reaction.

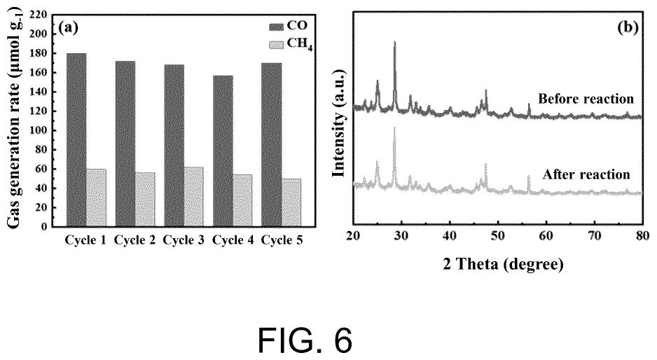

### DETAILED DESCRIPTION OF THE EMBODIMENTS

In the following, this application explains the technical scheme in detail through specific embodiments, but these embodiments should be clearly put forward for illustration, but they are not interpreted as limiting the scope of this application.

The photocatalyst activity evaluation is as follows. The CO₂ reduction activity of Bi₂S₃/ZnS composites is tested in a 100 mL stainless steel reactor with a 300 W xenon lamp as the light source and a working current of 6 A. The composite material (10 mg) is uniformly dispersed in quartz glass, 18 mL deionized water is used as solvent, and 2 mL TEOA is used as sacrificial agent. After 5 mins of ultrasonic vibration, it is transferred to a stainless steel reactor. Before the photoreduction of CO₂ experiment, the high purity CO₂ gas (99.995%) is used to ventilate for 2 h, and the air in the reactor is discharged. Then the valve at one end of the reactor is closed, carbon dioxide is passed to a pressure of 4 bar. The xenon lamp is turned on, and samples are taken and analyzed at intervals of 1 h during the illumination process, and gas products are detected by gas chromatography (GC-2030).

### Embodiment 1

This embodiment proposes a Bi₂S₃/ZnS composite comprising S—Bi double defect, the preparation method of which includes the steps as follows.

2 mmol bismuth nitrate pentahydrate was weighed into beaker A. 30 mL 0.1M mannitol solution measured into beaker A, which marked as A solution. And then the A solution was stirred at room temperature for 1 hour until it is clear.

3 mmol zinc chloride was weighed into beaker B. 20 mL deionized water was measured into the beaker B, which marked as B solution. The solution was ultrasonicized for 30 min, and then stirred at room temperature for 30 min.

The A solution was poured into the B solution and stirred for 10 min. 25 mmol L-cysteine was weighed into a beaker and stirred for 1 h. Then the stirred mixture was transferred to a hydrothermal kettle and sealed, placed in an oven, and reacted at 180° C. for 22 h. After the reactant was cooled to room temperature, the precipitate in the reactor was taken out and washed several times with deionized water and ethanol. The obtained product was dried in an oven at 60° C. for 12 h.

### Embodiment 2

This embodiment proposes a Bi₂S₃/ZnS composite comprising S—Bi double defect. The difference between the preparation method of this embodiment and the Embodiment 1 is that, the amount of zinc chloride is adjusted from “3 mmol” to “0.3 mmol”, and the other steps and parameters are the same as those in the Embodiment 1.

### Embodiment 3

This embodiment proposes a Bi₂S₃/ZnS composite comprising S—Bi double defect. The difference between the preparation method of this embodiment and the Embodiment 1 is that, the amount of zinc chloride is adjusted from “3 mmol” to “6 mmol”, and the other steps and parameters are the same as those in the Embodiment 1.

### Embodiment 4

This embodiment proposes a Bi₂S₃—ZnS composite comprising S—Bi double defect. The difference between the preparation method of this embodiment and the Embodiment 1 is that, the concentration of mannitol solution is adjusted from “0.1M” to “0.5 mmol”, and the other steps and parameters are the same as those in the Embodiment 1.

### Comparative Example 1

This embodiment proposes a Bi₂S₃/ZnS composite. The difference between the preparation method of this embodiment and the Embodiment 1 is that, the solvent is adjusted from “mannitol solution” to “ethylene glycol solution”, and the other steps and parameters are the same as those in the Embodiment 1.

### Comparative Example 2

This embodiment proposes a Bi₂S₃/ZnS composite. The difference between the preparation method of this embodiment and the Embodiment 1 is that, the solvent is adjusted from “mannitol solution” to “potassium hydroxide solution”, and the other steps and parameters are the same as those in the Embodiment 1.

### Comparative Example 3

This embodiment proposes a Bi₂S₃/ZnS composite. The difference between the preparation method of this embodiment and the Embodiment 1 is that, the solvent is adjusted from “mannitol solution” to “sodium hydroxide solution”, and the other steps and parameters are the same as those in the Embodiment 1.

### Comparative Example 4

This embodiment proposes a ZnS Photocatalyst, the synthesis method of which includes the steps as follows.

3 mmol of zinc chloride was dissolved in 20 mL of deionized water. The solution was placed in an ultrasonic cleaner ultrasound for 30 min, then stirred at room temperature for 30 min, until the solution turned milky white. Then 25 mmol L-cysteine was poured into the solution and stirred at room temperature for 1 h until the solution was transparent. The mixed solution was transferred to a sealed PTFE-lined stainless steel autoclave and reacted in an oven at 180° C. for 22 hours. After the reactant was cooled to room temperature, the precipitate in the reactor was taken out and washed several times with deionized water and ethanol. The obtained product was dried in an oven at 60° C. for 10 h to obtain a ZnS sample.

### Comparative Example 5

This embodiment proposes a D-Bi₂S₃ Photocatalyst, the synthesis method of which includes the steps as follows.

0.05 mol mannitol was dissolved in 500 mL deionized water, ultrasonicized for 30 min, and configured into 0.1 M mannitol solution. 2 mmol bismuth nitrate pentahydrate was weighed in a beaker. 30 mL 0.1M mannitol solution was taken and slowly poured into the beaker, and stirred for 1 h at room temperature to clarify it. Then 25 mmol L-cysteine was poured into the mixed solution and stirred at room temperature for 1 h until the solution was green and transparent. The mixed solution was then transferred to a 100 mL PTFE-lined stainless steel autoclave sealed and reacted in an oven at 180° C. for 22 hours. After the reactant was cooled to room temperature, the precipitate in the reactor was taken out and washed several times with deionized water and ethanol. The obtained product was dried in an oven at 60° C. for 12 h to obtain a D-Bi₂S₃ sample.

### Comparative Example 6

This embodiment proposes a M-Bi₂S₃ Photocatalyst, the synthesis method of which includes the steps as follows.

2 mmol Bi (NO₃)₃·5H₂O was dissolved in 30 mL ethylene glycol, and the solution was magnetically stirred at room temperature for 1 h to form a transparent solution. Then 25 mmol L-cysteine was poured into the solution and stirred at room temperature for 1 h until the solution was green and transparent. The mixed solution was then transferred to a 100 mL PTFE-lined stainless steel autoclave sealed and reacted in an oven at 180° C. for 22 hours. After the reactant was cooled to room temperature, the precipitate in the reactor was taken out and washed several times with deionized water and ethanol. The obtained product was dried in an oven at 60° C. for 12 h to obtain a M-Bi₂S₃ sample.

The photocatalytic activity of the composites and photocatalysts obtained by the above embodiments and the Comparative Examples was tested. The yields of the reduced products CO and CH₄ were measured after 5 hours of illumination, as shown in Table 1.

TABLE 1
The yields of the reduced products CO and CH₄

| | The yield of | The yield of |
|---|---|---|
| | CO/μmol · g⁻¹ | CH₄/μmol · g⁻¹ |
| Embodiment 1 | 179.7 | 57.89 |
| Embodiment 2 | 82.45 | 25.65 |
| Embodiment 3 | 96.15 | 35.4 |
| Embodiment 4 | 70.69 | 33.0 |
| Comparative Example 1 | 81.00 | 61.5 |
| Comparative Example 2 | 122.45 | 34.2 |
| Comparative Example 3 | 107.26 | 41.77 |
| Comparative Example 4 | 73.11 | 19.9 |
| Comparative Example 5 | 78.3 | 26.7 |
| Comparative Example 6 | 43.34 | 16.88 |

It can be seen from the contrast of the above data that, the solvent is replaced by ethylene glycol (Comparative Example 1), potassium hydroxide (Comparative Example 2) and sodium hydroxide (Comparative Example 3) described in the Comparative Example s to the same hydroxyl-containing mannitol described in this application, the photocatalytic activity of the resulting Bi₂S₃/ZnS composites is greatly improved. In particular, the yield of CO of the Embodiment 1 was increased by 122% compared to the Comparative Example 1, which was much higher than expected. This indicates that the choice of solvent has an important influence on the photocatalytic performance of the composites during the preparation of Bi₂S₃/ZnS composites comprising S—Bi double defect.

The above is only the preferred embodiment of the present application, but the scope of protection of the present application is not limited thereto, and any equivalents or modifications of the technical solutions of the present application and the application concept thereof should be comprised in the scope of the present application within the scope of the technical scope of the present application.

1. A preparation method of Bi₂S₃/ZnS composites comprising S—Bi double defect, comprising: dissolving bismuth nitrate pentahydrate in a mannitol solution to obtain a solution A; dissolving zinc chloride in water to obtain a solution B; mixing the solution A with the solution B, adding L-cysteine, and then obtaining the Bi₂S₃/ZnS composites by a hydrothermal reaction.

2. The preparation method of Bi₂S₃/ZnS composites comprising S—Bi double defect according to claim 1, wherein, in the solution A, a molar concentration of the bismuth nitrate pentahydrate is 1-5 mmol:30 ml.

3. The preparation method of Bi₂S₃/ZnS composites comprising S—Bi double defect according to claim 1, wherein, a concentration of the mannitol solution is 0.1-0.5M.

4. The preparation method of Bi₂S₃/ZnS composites comprising S—Bi double defect according to claim 1, wherein, in the solution B, a molar concentration of the zinc chloride is 0.3-6 mmol:20 ml.

5. The preparation method of Bi₂S₃/ZnS composites comprising S—Bi double defect according to claim 1, wherein, a molar ratio of the bismuth nitrate pentahydrate to the zinc chloride is 2:0.3-6, and a molar ratio of bismuth nitrate pentahydrate to L-cysteine is 2:20-30.

6. The preparation method of Bi₂S₃/ZnS composites comprising S—Bi double defect according to claim 1, wherein, a molar ratio of the bismuth nitrate pentahydrate to the zinc chloride is 2:3, and a molar ratio of the bismuth nitrate pentahydrate to the L-cysteine is 2:25.

7. The preparation method of Bi₂S₃/ZnS composites comprising S—Bi double defect according to claim 1, wherein, the hydrothermal reaction temperature is 160-200° C., and the hydrothermal reaction time is 18-26 h.

8. The preparation method of Bi₂S₃/ZnS composites comprising S—Bi double defect according to claim 1, wherein, further comprising washing and drying products of the hydrothermal reaction, the drying temperature is 50-80° C. and the drying time is 10-20 h.

9. The preparation method of Bi₂S₃/ZnS composites comprising S—Bi double defect according to claim 1, wherein, the zinc chloride is dissolved in water and ultrasonicized for 10-40 min, and stirred for 10-40 min to obtain the solution B.

10. A Bi₂S₃/ZnS composite comprising S—Bi double defect prepared by the method of claim 1.
